(19) United States
(12) Patent Application Publication
(10) Pub. No.: US 2023/0255481 A1
(43) Pub. Date: Aug. 17, 2023

(54) Electronic Pressure Sensor for Eye with Optical Interface

(71) Applicant: Twenty Twenty Therapeutics LLC, South San Francisco, CA (US)

(72) Inventors: Supriyo Sinha, Menlo Park, CA (US); Oleg Rumyantsev, Menlo Park, CA (US); Jeremy Chan, San Jose, CA (US); Dimitri Azar, Chicago, IL (US)

(21) Appl. No.: 18/158,365

(22) Filed: Jan. 23, 2023

Related U.S. Application Data
(60) Provisional application No. 63/306,000, filed on Feb. 2, 2022.
(60) Provisional application No. 63/306,002, filed on Feb. 2, 2022.

(51) Int. Cl. A61B 3/16

(52) U.S. Cl. CPC A61B 3/16; A61B 2560/0214; A61B 2562/0247; A61B 2562/028

(57) ABSTRACT

An intraocular pressure sensing element along with implant microelectronic circuitry to be configured to be implanted into an eye of a user. The microelectronic circuitry is conductively coupled to the intraocular pressure sensing element to produce measured pressure data. A microscopic light emitting diode, LED, that is also implanted into the eye, is driven with the measured pressure data thereby optically transmitting the measured pressure data for communication with outside of the eye. A photovoltaic element that is also implanted into the eye supplies energy to operate the implant microelectronic circuitry and the microscopic LED. Other aspects are also described and claimed.

This nonprovisional patent application claims the benefit of the earlier filing dates of U.S. provisional patent applications 63/306,000 and 63/306,002 both filed 2 Feb. 2022.

### FIELD

The subject matter of this disclosure relates to techniques for measuring intraocular pressure in human eyes, using an implantable pressure sensor. Such techniques are useful for treating and/or monitoring progression of eye diseases including glaucoma, but are not limited to use with the treatment of eye disease.

### BACKGROUND

Intraocular pressure (IOP) refers to the pressure of a fluid known as the aqueous humor inside the eye. The pressure is normally regulated by changes in the volume of the aqueous humor, but some individuals suffer from disorders, such as glaucoma, that cause chronic heightened IOP. Over time, heightened IOP can cause damage to the eye's optical nerve, leading to loss of vision. Presently, treatment of glaucoma mainly involves periodically administering pharmaceutical agents to the eye to decrease IOP. These drugs can be delivered, for example, by injection or eye drops. However, effective treatment of glaucoma requires adherence to dosage schedules and a knowledge of the patient's IOP. The more current or recent the measurement is, the more relevant it will be and hence the more effective the resulting treatment can be. The IOP for a given patient can vary significantly based on time of day, exercise, how recently a medication was taken, and other factors. Typically, IOP measurements are performed in a doctor's office and often no more than once or twice per year. These infrequent measurements are less able to account for variation in the patient's IOP, and may become stale due to the length of time between them. This means that any given measurement is subject to uncertainty, so it may take several IOP measurements over time to have confidence in the health of the patient's eye.

Typically, the IOP is measured using a tonometer, which is a device that is outside the eye and thus does not require a sensor within the eye. Contact tonometry is performed in a clinical setting, and the procedure requires numbing of the patient's eye, resulting in both inconvenience and discomfort. Noncontact tonometry involves directing a puff or jet of air towards the patient's eye and measuring the resulting deflection dynamics of the cornea. However, this requires a bulky and power hungry pump arrangement that may not be practical for home use, and is not as accurate as contact tonometry.

A wireless, implantable, continuous IOP monitoring system has been suggested that has a commercial pressure sensing element with digital readout, and a microelectronic chip that supports wireless power/data telemetry and a wired serial communication interface with the pressure sensing element. An on-chip integrated RF coil receives power from near-field RF coupling at 915 MHz, and transmits pressure measurement bits via RF-backscattering to an external reader.

### SUMMARY

An aspect of the disclosure here is an intraocular pressure, IOP, sensor that, once implanted into an eye of a user, may be powered optically through ambient lighting (e.g., by sunlight, room or office lighting) rather than by a dedicated light source or by any specific action of its user. The IOP sensor includes an implanted intraocular pressure sensing element that is coupled to implant microelectronic circuitry. The intraocular pressure sensing element may be implanted into the cornea or sclera, without piercing into the anterior chamber of the eye. Also implanted into the cornea or sclera may be the implant microelectronic circuitry, which may be implanted into an area of the sclera that is comparable in thickness to the cornea. The implant microelectronic circuitry produces measured pressure data, using the intraocular pressure sensing element. The implant microelectronic circuitry communicates the measured pressure data optically, e.g., via a microscopic light emitting diode (micro LED) that transmits the data out of the eye. In addition to the implanted microelectronic circuitry and the implanted intraocular pressure sensing element, the IOP sensor also includes an implanted photovoltaic element which supplies energy to operate other components of the IOP sensor. The IOP sensor is thus said to have an all optical interface which includes the micro LED and the photovoltaic element, and that enables the use of light to both power the IOP sensor and communicate measured pressure data out of the IOP sensor. The all optical interface enables the IOP sensor to be small, e.g., less than ten cubic millimeters in volume, as compared to an implantable IOP sensor that has an RF data communication antenna. Such a small size reduces interference with operation of the eye and reduces surgical side effects, while the IOP sensors ability to be powered by ambient light alone makes the system as whole, for measuring IOP, easier for the user to use.

For the optical interface to enjoy greater optical transparency, the micro LED and the photovoltaic element can be implanted into the cornea. However, the micro LED and the photovoltaic element could alternatively be implanted into the sclera so long as the higher light scattering characteristics of the sclera are taken into consideration when designing the IOP sensor as a whole. Aspects of the IOP sensor to consider here include power consumption of the implant microelectronic circuitry, energy capacity and size of a battery that temporarily stores the energy produced by the photovoltaic element, and the power output and size of the photovoltaic element. Placing as much the IOP sensor as possible into the sclera could reduce risk of complications for the vision of the user. The fidelity of sensing the intraocular pressure (by the intraocular pressure sensing element) would depend on the implantation depth of the intraocular pressure sensing element and may calibrated after surgery.

The optically transmitted measured pressure data is received by outside-of-the-eye microelectronic circuitry that is integrated into a handheld or portable reader device. The reader device can be held by the user, and when brought close to the user's eye it can receive the optically transmitted measured pressure data. The received measured pressure data may then be further processed or evaluated by the outside-of-the-eye microelectronic circuitry, for purposes of for example informing the user about their intraocular pressure.

The above summary does not include an exhaustive list of all aspects of the present disclosure. It is contemplated that the disclosure includes all systems and methods that can be practiced from all suitable combinations of the various aspects summarized above, as well as those disclosed in the Detailed Description below and particularly pointed out in the Claims section. Such combinations may have particular advantages not specifically recited in the above summary.

### BRIEF DESCRIPTION OF THE DRAWINGS

Several aspects of the disclosure here are illustrated by way of example and not by way of limitation in the figures of the accompanying drawings, in which like references indicate similar elements. It should be noted that references to “an” or “one” aspect in this disclosure are not necessarily to the same aspect, and they mean at least one. Also, in the interest of conciseness and reducing the total number of figures, a given figure may be used to illustrate the features of more than one aspect of the disclosure, and not all elements in the figure may be required for a given aspect.

FIG. 1A shows an example system for measuring intraocular pressure, using a reader device and a cornea implanted IOP sensor having an optical power interface and an optical data interface.

FIG. 1B shows an example system for measuring intraocular pressure in which the IOP sensor, having an optical power interface and an optical data interface, is implanted into sclera.

FIG. 2 depicts a user holding the reader device close to their eye.

FIG. 3 is a block diagram illustrating an example system for measuring intraocular pressure.

### DETAILED DESCRIPTION

Several aspects of the disclosure with reference to the appended drawings are now explained. Whenever the shapes, relative positions and other aspects of the parts described are not explicitly defined, the scope of the invention is not limited only to the parts shown, which are meant merely for the purpose of illustration. Also, while numerous details are set forth, it is understood that some aspects of the disclosure may be practiced without these details. In other instances, well-known circuits, structures, and techniques have not been shown in detail so as not to obscure the understanding of this description.

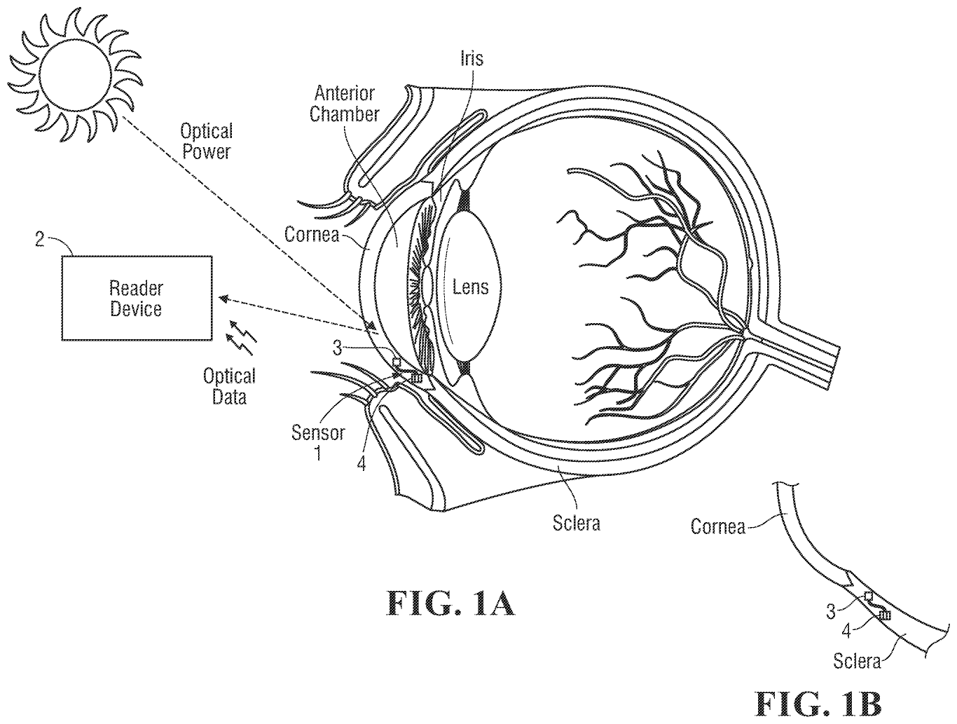

FIG. 1A and FIG. 1B show an example system for measuring intraocular pressure, using a reader device 2 and an implanted IOP sensor 1. The IOP sensor 1 is composed of several parts or components which cooperate to enable the sensor 1 to have an all optical interface, e.g., an optical power interface and an optical data interface. Note that some of these parts are depicted in a block diagram of the system shown in FIG. 3. In the FIG. 1A, the IOP sensor is implanted in its entirety within the cornea, while in FIG. 1B the IOP sensor is implanted in its entirety within the sclera.

The IOP sensor 1 includes the following components. An intraocular pressure sensing element 3 is to be implanted into an eye of a user, in the cornea (e.g., as shown in FIG. 1A) or in the sclera (e.g., as shown in FIG. 1B.) The intraocular pressure sensing element 3 may be, for example, a capacitive or piezoelectric pressure sensing element. The pressure sensing element is conductively coupled to implant microelectronic circuitry 4 which is also implanted into the eye, in the cornea as shown in the example of FIG. 1A or in the sclera as shown in the example of FIG. 1B. This conductive coupling may be through a rigid connection between a housing of the intraocular pressure sensing element 3 and a housing of the implant microelectronic circuitry 4; the rigid connection could simplify surgical implantation of the IOP sensor 1. Alternatively, the connection may be a flexible one like a tether which allows the two components (the intraocular pressure sensing element 3 and the housing that contains the implant microelectronic circuitry 4 and perhaps other components described below) to be positioned at separately optimal locations in the eye.

The implant microelectronic circuitry 4 uses a signal (in the conductive coupling) from the intraocular pressure sensing element 3 to produce measured pressure data. The signal may be an analog signal (e.g., that is input to a sensing amplifier which is part of the implant microelectronic circuitry 4), or it may be a digital signal (e.g., the sensing amplifier and a digitizer are part of the intraocular pressure sensing element 3.) The implant microelectronic circuitry 4 drives a microscopic light emitting diode, micro LED 6, the latter also being implanted in the eye of the user for example within the same housing or package as the implanted microelectronic circuitry 4. The micro LED 6 is thus optically transmitting the measured pressure data, for communication with outside of the eye. The micro LED 6 may transmit light at a wavelength that is in the visible region or in the near infrared region. The micro LED 6 may be no more than 0.2 millimeters squared in area. The micro LED 6 may be one of several micro LEDs that are implanted into the eye and that are driven with the measured pressure data for communication with outside of the eye, e.g., a pair of micro LEDs that are operated in a different data transmission mode to improve signal to noise ratio particularly when the ambient light is changing.

The IOP sensor 1 also includes a photovoltaic element 7 which is also implanted into the eye, and may be within the same housing or package as the implant microelectronic circuitry 4. The photovoltaic element 7 is configured to convert incident ambient light, e.g., solar, into energy that it supplies to operate the implant microelectronic circuitry 4 and the microscopic LED 6. The photovoltaic element 7 may be configured to continuously provide its converted energy to be stored temporarily within, or recharge, a battery 8. The battery 8 is also implanted in the eye, for example within the same housing or package as the implant microelectronic circuitry 4 or the photovoltaic element 7, and supplies its stored energy to power the components of the IOP sensor 1. Note, the term “battery” is used generically here, to refer to any type of rechargeable solid state device (e.g., a solid state battery) or other electricity storage device that can be charged and recharged by the photovoltaic element 7. In one aspect, the photovoltaic element 7 is no more than 5 square millimeters in area, supporting a total power consumption by the IOP sensor 1 of a few tens of nano Watts on average. In one aspect, the photovoltaic element 7 should be tuned to also work (have a reasonable level of photovoltaic output power per area) with solely indoor ambient lighting, such as in a warehouse, office space, or retail space. The photovoltaic element 7 and the micro LED 6 should be placed in a region of the eye that that has sufficient optical transparency to the outside world for them to operate as intended, for example in the cornea as shown in FIG. 1A or in the sclera as shown in FIG. 1B.

In one aspect, there may be a single package that contains the battery 8, the photovoltaic element 7, the implant microelectronic circuitry 4 and the micro LED 6, and such a package may be less than 10 cubic millimeters in volume. This size may be largely dictated by the size of the battery 8 and the photovoltaic element 7.

In one aspect, the implant microelectronic circuitry 4 may include an application specific integrated circuit, ASIC, that has a watchdog timer that triggers the taking of measurements of the IOP (or producing measured pressure data) at intervals programmed into the watchdog timer. These intervals may be regular (e.g., one measurement every fifteen minutes) or they may be irregular (e.g., one measurement every half hour, and once a day a burst of measurements is taken every five hundred milliseconds for ten seconds.) Each measurement may be stored in memory of the implant microelectronic circuitry 4 (e.g., in a static random access memory module) along with a timestamp for the measurement, until it is time to transmit the measurement (as produced measured pressure data) out of the IOP sensor 1. The capacity of the battery 8 should be selected to provide enough energy for taking such measurements even when the user's eye (in which the IOP sensor 1 has been implanted) has not been exposed to light that can recharge the battery 8 (e.g., solar light outside), for a reasonable amount of time. This reasonable amount of time, during which only the energy stored in the battery 8 will power such measurements, is between seven hours (which is close to the average recommended hours of sleep per day for an adult) and thirty-six hours, e.g., twenty-four hours. In one aspect, this allows the IOP sensor 1 to take measurements at night time or when the user is asleep.

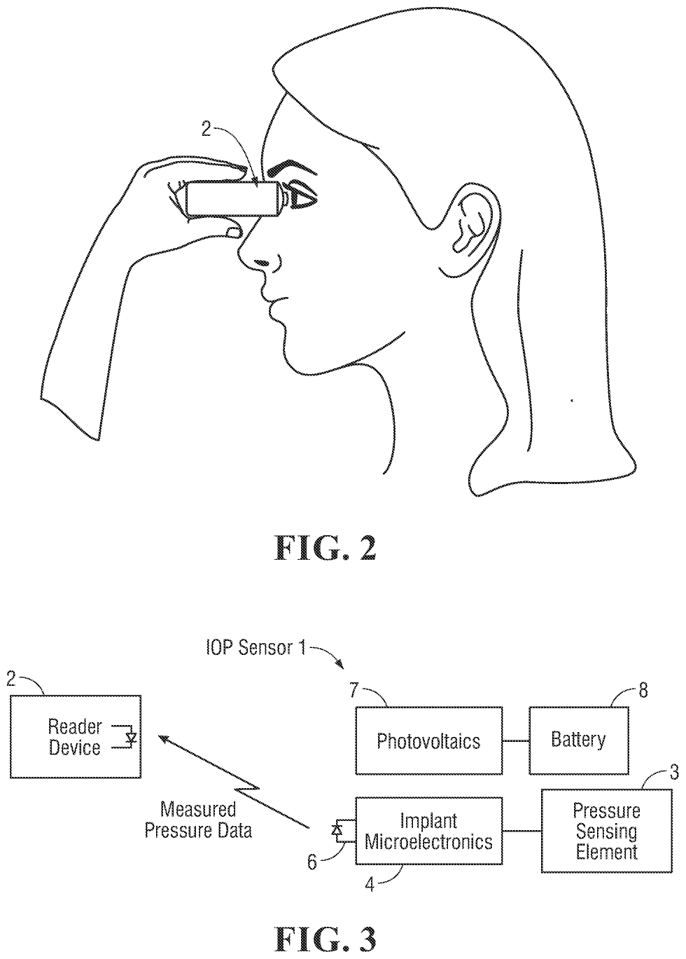

FIG. 2 depicts a user holding the reader device 2 in their hand, and who has brought the reader device close to their eye. In this aspect, the reader device 2 is a portable or handheld device in which outside-the-eye microelectronic circuitry has been integrated. The outside-the-eye microelectronic circuitry is configured to receive the transmitted, measured pressure data once the reader device 2 is close to the eye, for example using or more LEDs or other suitable photodetector arrangement. It then processes the received measured pressure data for informing the user about their IOP. The reader device 2 is deemed close to the eye when it can receive the optically transmitted, measured pressure data, for example at no more than three inches away from the eye.

In one aspect, the outside-the-eye microelectronic circuitry transmits an optical interrogation signal that is detected by the implant microelectronic circuitry using the microscopic LED (operating in reverse bias), the photovoltaic element, or a separate photodetector element. In response, the implant microelectronic circuitry becomes aware that the reader device 2 is within range to receive from the IOP sensor 1, and therefore drives the micro LED with the measured pressure data thereby optically transmitting the measured pressure data for communication with the outside-of the-eye microelectronic circuitry.

In one aspect, the transmit power of the micro LED is adjusted so that a maximum amount of measured pressure data can be transmitted or uploaded, with the available energy in the battery 8. In another aspect, the optical receiver circuitry of the outside-the-eye microelectronic circuitry is optimized so as to maximize signal to noise ratio (e.g., via spectral filtering.) Also, error correction bits may be included in the transmitted, measured pressure data, which the outside-the-eye microelectronic circuitry uses to ensure that it is receiving the pressure data correctly. Bi-directional communication between the outside-the-eye microelectronic circuitry and the implant microelectronic circuitry may help further ensure that IOP sensor 1 aware of the fact that the measured pressure data has been uploaded into the reader device 2. In a further aspect, the memory within the IOP sensor 1 has sufficient data storage capacity to store or log all measurements that are taken between measured pressure data upload times.

In yet a further aspect, the implant microelectronic circuitry operates predominantly, e.g., more than 99% of the time, in a background mode of operation in which it produces no measured pressure data and in which it transmits no measured pressure data. This helps reduce its power consumption, which in turns reduces the size of the photovoltaic element and the battery.

In one aspect, the IOP sensor 1 may be configured to be used as an on-demand device: in a first phase of an interaction between the reader device 2 and the IOP sensor 1, the reader device 2 provides power to the IOP sensor 1; and then in a second phase the IOP sensor 1 takes the measurements and transmits the measured pressure data back to the reader device 2.

While certain aspects have been described and shown in the accompanying drawings, it is to be understood that such are merely illustrative of and not restrictive on the broad invention, and that the invention is not limited to the specific constructions and arrangements shown and described, since various other modifications may occur to those of ordinary skill in the art. For example, while FIG. 1A and FIG. 1B illustrate the transmitter and receiver as photodiode symbols, other types of photo-emitters and photo-detectors may be used in the transmitter and the receiver. Also, whereas those figures show the Sun as providing its sunlight to power the IOP sensor 1, other sources of optical power are possible such as artificial room lighting or a lamp that is within an accessory worn by the user (e.g., eye glasses.) The description is thus to be regarded as illustrative instead of limiting.

What is claimed is:

1. An intraocular electronic pressure sensor comprising:
an intraocular pressure sensing element configured to be implanted into an eye of a user;
implant microelectronic circuitry to be implanted into the eye of the user and conductively coupled to the intraocular pressure sensing element to produce measured pressure data;
a microscopic light emitting diode, microscopic LED, to be implanted into the eye of the user, wherein the implant microelectronic circuitry is to drive the microscopic LED with the measured pressure data thereby optically transmitting the measured pressure data for communication with outside of the eye; and
a photovoltaic element to be implanted into the eye of the user, wherein the photovoltaic element is to supply energy to operate the implant microelectronic circuitry and the microscopic LED.

2. The intraocular electronic pressure sensor of claim 1 wherein one or more of the intraocular pressure sensing element, the microelectronic circuitry, the microscopic LED and the photovoltaic element are configured to be implanted into a cornea or sclera.

3. The intraocular electronic pressure sensor of claim 1 wherein the microscopic LED is one of a plurality of microscopic LEDs to be implanted into the eye of the user and that are driven with the measured pressure data for communication with the outside of the eye.

4. The intraocular electronic pressure sensor of claim 1 wherein the implant microelectronic circuitry is conductively coupled to the intraocular pressure sensing element via a flexible connection or via a rigid connection.

5. The intraocular electronic pressure sensor of claim 4 in combination with outside-of-the-eye microelectronic circuitry that is configured to receive the transmitted, measured pressure data, and process the received data for informing the user about their intraocular pressure.

6. The intraocular electronic pressure sensor of claim 5 wherein the outside-of-the-eye microelectronic circuitry is integrated into a portable or handheld device and can receive the transmitted data when the portable or handheld device is held by the user close to their eye.

7. The intraocular electronic pressure sensor of claim 6 wherein the outside-of-the-eye microelectronic circuitry is to transmit an optical interrogation signal that is detected by the implant microelectronic circuitry using the microscopic LED, the photovoltaic element, or a separate photodetector element, and in response the implant microelectronic circuitry drives the microscopic LED with the measured pressure data thereby optically transmitting the measured pressure data for communication with the outside-of the-eye microelectronic circuitry.

8. The intraocular electronic pressure sensor of claim 1 further comprising a rechargeable battery to be implanted into the eye of the user and configured to store the energy supplied by the photovoltaic element.

9. The intraocular electronic pressure sensor of claim 8 wherein the implant microelectronic circuitry operates predominantly in a background mode of operation in which no measured pressure data is produced and no measured pressure data is transmitted.

10. The intraocular electronic pressure sensor of claim 9 in combination with outside-of-the-eye microelectronic circuitry that is configured to receive the transmitted, measured pressure data, and process the received data for informing the user about their intraocular pressure.

11. The intraocular electronic pressure sensor of claim 10 wherein the outside-of-the-eye microelectronic circuitry is integrated into a portable or handheld device and can receive the transmitted data when the portable or handheld device is held by the user close to their eye.

12. The intraocular electronic pressure sensor of claim 11 wherein the outside-of-the-eye microelectronic circuitry is to transmit an optical interrogation signal that is detected by the implant microelectronic circuitry using the microscopic LED, the photovoltaic element, or a separate photodetector element, and in response the implant microelectronic circuitry drives the microscopic LED with the measured pressure data thereby optically transmitting the measured pressure data for communication with the outside-of the-eye microelectronic circuitry.

13. The intraocular electronic pressure sensor of claim 1 wherein the implant microelectronic circuitry operates predominantly in a background mode of operation in which no measured pressure data is produced and no measured pressure data is transmitted.

14. The intraocular electronic pressure sensor of claim 1 in combination with outside-of-the-eye microelectronic circuitry that is configured to receive the transmitted, measured pressure data, and process the received data for informing the user about their intraocular pressure.

15. The intraocular electronic pressure sensor of claim 14 wherein the outside-of-the-eye microelectronic circuitry is integrated into a portable or handheld device and can receive the transmitted data when the portable or handheld device is held by the user close to their eye.

16. The intraocular electronic pressure sensor of claim 15 wherein the outside-of-the-eye microelectronic circuitry is to transmit an optical interrogation signal that is detected by the implant microelectronic circuitry using the microscopic LED, the photovoltaic element, or a separate photodetector element, and in response the implant microelectronic circuitry drives the microscopic LED with the measured pressure data thereby optically transmitting the measured pressure data for communication with the outside-of the-eye microelectronic circuitry.

17. A method for intraocular pressure monitoring using an electronic intraocular pressure sensor that is implanted in an eye of a user and a reader device that is outside the eye, the method comprising:
converting, by a photovoltaic element of the sensor, ambient light that is incident on the eye of user into electrical energy that powers the sensor;
transmitting, by a microscopic LED of the sensor, the measured pressure data;
receiving, by a photodetector of the reader device, the transmitted, measured pressure data; and
processing the received data for informing the user about their intraocular pressure.

18. The method of claim 17 further comprising
transmitting, by the reader device, an optical interrogation signal that is detected by the sensor, wherein transmitting the measured pressure data by the micro LED is in response to having detected by the optical interrogation signal.

19. The method of claim 18 further comprising
providing optical power by the reader device to charge a battery of the sensor in a first phase of an interaction between the sensor and the reader device, wherein transmitting by the sensor the measured pressure data occurs second phase of the interaction and only in response to the sensor being charged during the first phase.

20. The method of claim 17 further comprising
providing optical power by the reader device to charge a battery of the sensor in a first phase of an interaction between the sensor and the reader device, wherein transmitting by the sensor the measured pressure data occurs second phase of the interaction and only in response to the sensor being charged during the first phase.
